(19) United States
(12) Patent Application Publication
(10) Pub. No.: US 2004/0188303 A1
(43) Pub. Date: Sep. 30, 2004

(54) Adjustable width container for coverslips

(76) Inventor: Richard J. Chouinard, Dover, NH (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 10/806,728

(22) Filed: Mar. 23, 2004

Related U.S. Application Data
(60) Provisional application No. 60/457,470, filed on Mar. 25, 2003.

(51) Int. Cl. B65D 85/48; C12M 1/34

(52) U.S. Cl. 206/456; 435/287.1

(57) ABSTRACT

A container for coverslips is disclosed wherein the length and/or width of the internal receptacle area is adjustable by repositioning one or more partitions.

### CROSS-REFERENCE TO RELATED APPLICATION

The present application is based on and claims priority from U.S. Provisional Patent Application Serial No. 60/457,470 filed on Mar. 25, 2003.

### FIELD OF THE INVENTION

The present invention relates to containers for holding a plurality of cover slips.

### BACKGROUND OF THE INVENTION

Coverslips are generally manufactured in standard sizes, for example, in widths of 22 mm or 24 mm, and lengths in the range of 22 mm to 60 mm, and are typically made of glass or plastic. The standard method of packaging coverslips is to place them into containers that can withstand the rigors of shipping and handling. Such containers are made to fit a particular coverslip size in order to prevent unwanted movement of coverslips within the container that could result in damage to the coverslips. Customized packaging to meet sizing needs adds to the expense of coverslip products.

Further, a number of instruments exist for automatically applying coverslips of either glass or plastic to microscopes slides. Examples of automatic devices for applying coverslips are described in U.S. Pat. No. 3,833,449 to Johnson, U.S. Pat. No. Pat. No. 3,930,928 to Tapert, and U.S. Pat. No. 3,972,423 to Tipton, all incorporated in entirety herein, as well as other automatic coverslipping instruments known in the art. Typically, an automatic coverslipper has a magazine or hopper-type container for holding a plurality of coverslips. Such hoppers are often durable structures made to fit one particular coverslip width, although some accommodate variation in the length of coverslips. Generally, different hoppers are used to fit each particular size of coverslips, especially when changing coverslip width, adding to the cost and inconvenience of using an automatic coverslipper. A need exists for a single container, of the disposable type or of the durable type, used for packaging, shipping, storage or with instruments, that can be adapted to hold a plurality of coverslips of any standard width and length.

### SUMMARY OF THE INVENTION

In one aspect, the invention comprises a container made of either a durable material or a material intended for disposal after limited use, and having means for adjusting the width and/or length of the interior container cavity to fit a variety of coverslip sizes. In another aspect, the invention comprises a method of adjusting the length and width of a container for holding a plurality of coverslips by strategic placement of one or more partition-like device(s) within the interior of the container.

Various other features, objects and advantages of the invention will be made apparent from the following description taken together with the drawings.

### BRIEF DESCRIPTION OF THE DRAWINGS

The drawings illustrate the best mode presently contemplated of carrying out the invention.

In the drawings:

FIG. 1 shows a perspective view of one embodiment of a coverslip container with partitions placed in position to size the receptacle for receiving 24 mm×40 mm coverslips;

FIG. 2 shows an exploded view of the embodiment of FIG. 1;

FIG. 3 shows a top view of one embodiment of a coverslip container with partitions placements indicated by dashed lines to size the receptacle for receiving 24 mm×40 mm, 24 mm×50 mm, or 24 mm×60 mm coverslips;

FIG. 4 shows a perspective view of one embodiment of a coverslip container with partitions in position to size the receptacle for receiving 22 mm×40 mm coverslips;

FIG. 5 shows an exploded view of the embodiment of FIG. 4.

FIG. 6 shows a top view of one embodiment of a coverslip container having had a partition placed to size the receptacle for receiving 22 mm×40 mm coverslips, as shown with dashed lines, altered by the placement of other partitions for converting the container to receive 24 mm×50 mm or 24 mm×60 mm coverslips;

FIG. 7 shows a perspective view of an embodiment featuring cut-out areas in the sidewalls for finger placement or for access by a robotic handler to facilitate the addition of coverslips into the receptacle area of the container;

FIG. 8 shows an exploded view of an embodiment wherein a single partition is used to adjust the length of the receptacle area of the container; and

FIG. 9 shows a top view of an embodiment with an aperture in the base of the container.

### DETAILED DESCRIPTION OF PREFERRED EMBODIMENTS

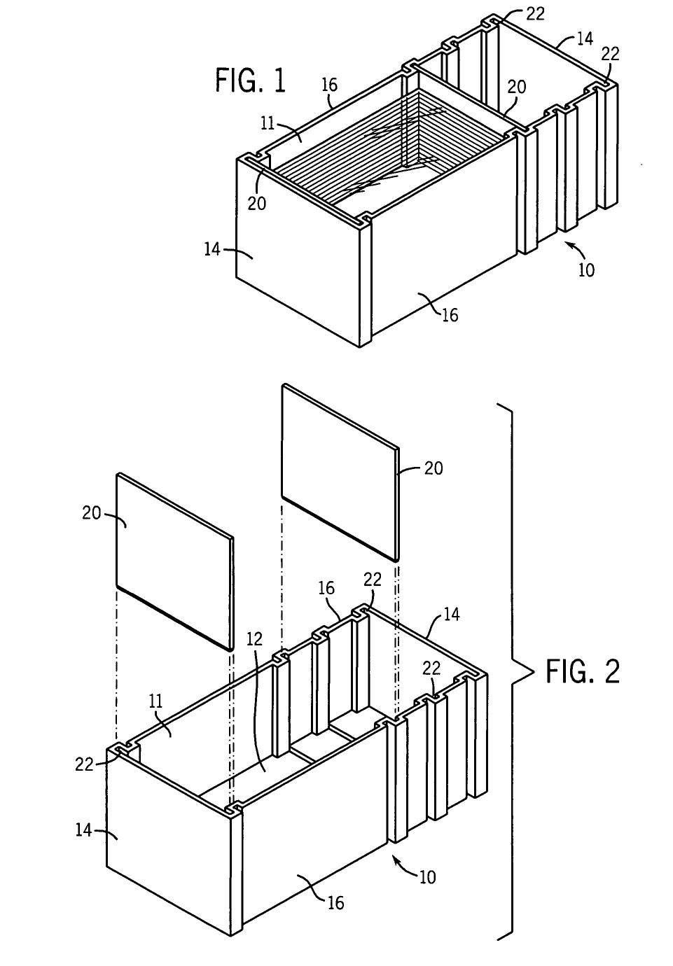

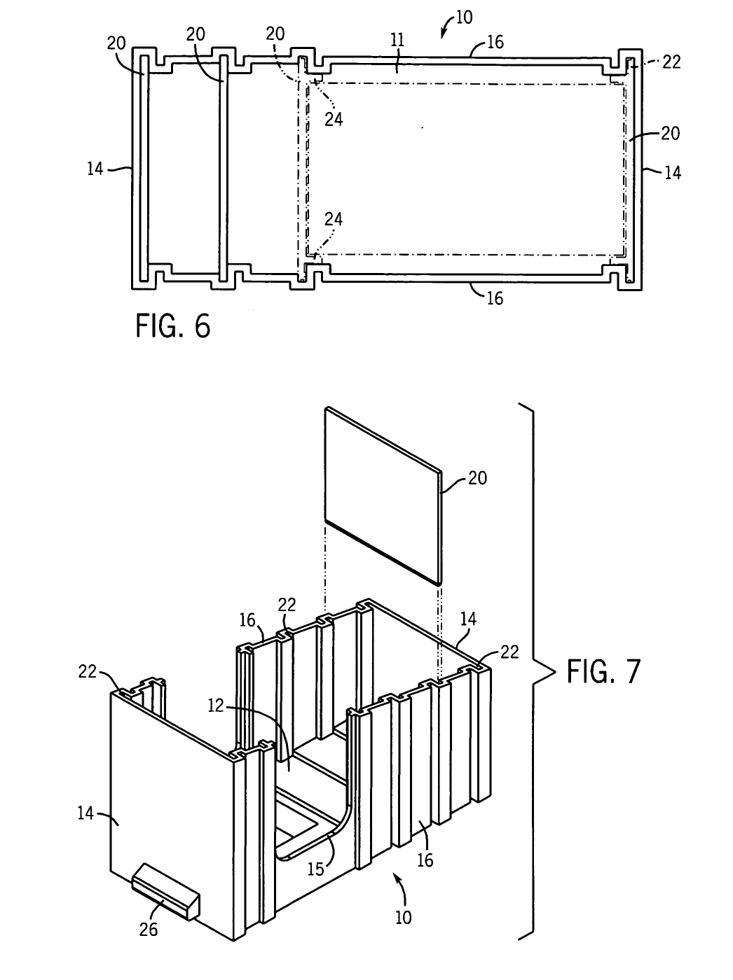

Referring to embodiments shown in FIGS. 1-9, the container 10 comprises a base 12, two opposed end walls 14 substantially perpendicular to the base 12, and two opposed, substantially perpendicular sidewalls 16 defining a receptacle portion 11 of the container 10. In one embodiment, the container 10 is made of an inexpensive disposable material, for example, polystyrene, polypropylene, or other suitable material. Alternatively, the container 10 is comprised of a more durable material suitable for repeated use, including, but not limited to acrylonitrile butadiene styrene (ABS), acetal, glass-filled nylon, aluminum, or the like. The container 10 is either molded, machined, or some combination, as is appropriate for the material selected. Also, the container 10 need not be fabricated of a single material; for example, the base 12, end walls 14 and/or sidewalls 16 can be made of different materials. The base 12, end walls 14, and/or sidewalls 16, if formed as separate parts, are glued, welded, force-fitted or joined by other methods to form the container 10. Depending upon the material used, the sidewalls 16 and/or end walls 14 of the container 10 are optionally reinforced with ribbing or other structural details.

Additional features can be included in the structure of the container 10 as appropriate for use in connection with a particular instrument. In one embodiment shown in FIG. 7, the sidewalls 16 are partially cut away to form openings 15 for finger placement or for access by a robotic handler to facilitate the addition of coverslips into the receptacle portion 11 of the container 10. Also shown in FIG. 7 is an optional guide means 26 to facilitate and/or maintain placement within an automatic instrument. Referring to FIG. 9, an aperture 28 is present in the base 12 of the container 10 to permit detection by sensory means of an automatic instrument of the presence or absence of coverslips in the container 10. Such features tend to be specific to the requirements of particular instruments are not material to the present invention.

Preferably, the length of the receptacle portion 11 is adequate to contain relatively long coverslips, for example, greater than or equal to 60 mm. Likewise, the width is sufficient to contain coverslips that are at least 24 mm in width. The depth of the container 10 is variable but is envisioned as sufficient to hold a plurality of coverslips, depending upon the thickness of the coverslips, for example at least 50 or more coverslips, but the container 10 can be made to hold hundreds of coverslips, for example approximately 500 coverslips.

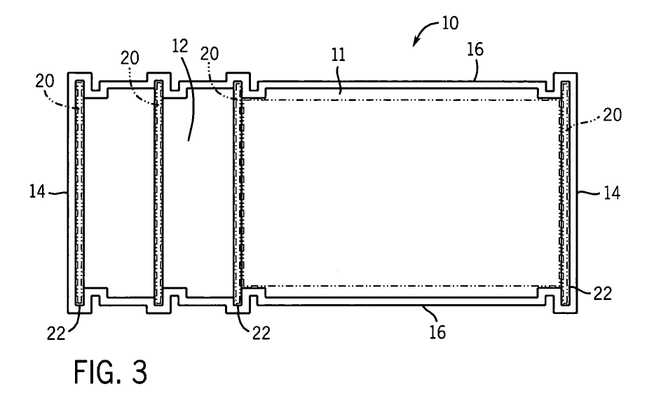

Referring to the embodiment shown in FIGS. 1-2, the length of the container 10 is adjusted by the placement of partitions 20, each comprising a substantially flat plate, within the receptacle portion 11 of the container 10. In some embodiments, slots and/or notches 22 in each of the opposing sidewalls 16 are used to facilitate placement of a partition 20. In an alternative embodiment not shown, the partition 20 is formed or made to fit securely against substantially flat sidewalls 16 within the receptacle 11 such that the partition 20 is not jarred out of place by movement of coverslips therein. The partition 20 is made of any suitable material such as polystyrene or polypropylene and the like. Optionally, a compressible material, such as rubber or foam, is used at the ends of the partition 20 to facilitate a snug fit against the sidewalls 16. FIG. 3 shows how partitions 20 can be used to adjust the receptacle portion 11 to fit various lengths of coverslips, for example in lengths of 40 mm, 50 mm, or 60 mm, by removal of the partition 20 from one placement cite to another; although FIG. 3 shows three partitions 20 at the variable end of the container 10, in use, only one partition 20 need be positioned to adjust the dimensions of the receptacle 11.

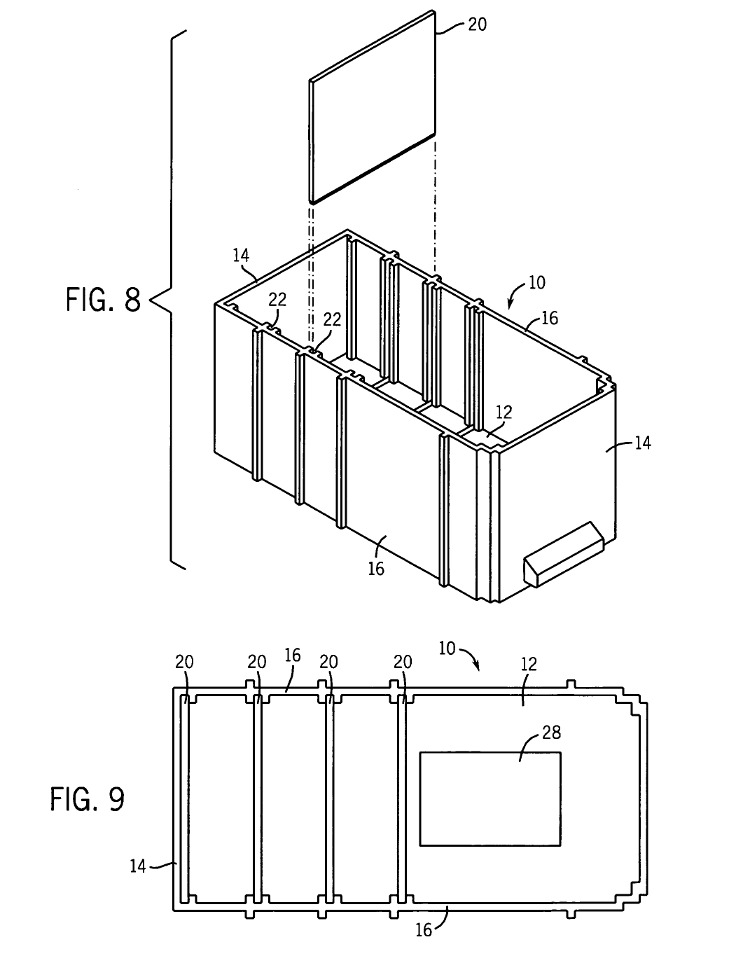

In the embodiments shown in FIGS. 1-7, the container 10 is structured to accept two partitions 20, with each defining opposite ends of the receptacle 11. Alternatively, the container 10 is designed so that only one partition 20 is utilized to define the receptable portion 11, as shown in FIGS. 8 and 9. In yet another embodiment, when the container 10 is made to accept two partitions 20, end walls 14 are not necessary and can be eliminated because the sidewalls 16 and partitions 20 can define the receptacle portion 11.

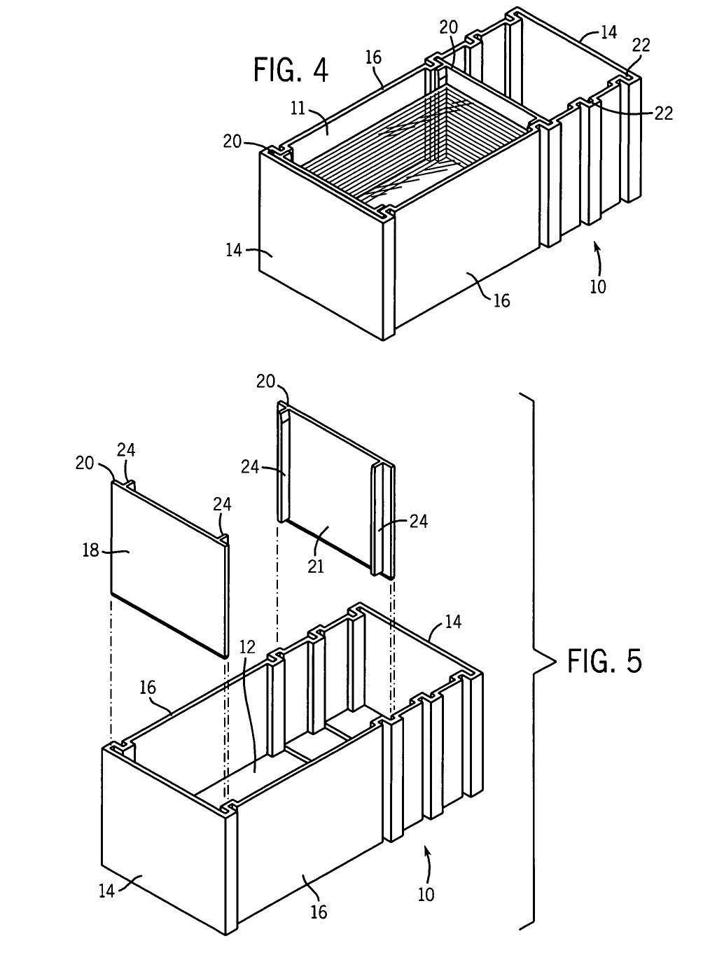

FIGS. 4 and 5 show one embodiment of a partition 20 useful for adjusting the width of the container 10 to fit coverslips with narrower widths, for example 22 mm widths. Instead of a simple flat plate, the partition 20 comprises a plate with one substantially flat side 18 while the opposite side 21 has projections 24 along or near one set of opposed peripheral edges of the partition 20 for abutting the sidewalls 16 of the container 10. Each projection 24 is approximately 1 mm in thickness to adjust the width from 24 mm to 22 mm, but other dimensions are envisioned to fit any range of coverslip widths. The projections 24 fit in slots or notches 22 in the sidewalls 16 of the receptacle portion 11 of the container 10 or, alternatively, the projections 24 fit snugly against substantially flat sidewalls 16. When the partition 20 is designed to fit into slots or notches 22 in the container sidewalls 16, the projections 24 are recessed inwardly from the edges of the partition 20 to provide means for fitting into the slot or notch 22. When the partition 20 is designed to fit against substantially flat sidewalls 16, the projections 24 are located at the periphery of a set of opposite edges of the partition 20. In an alternative embodiment, a single projection 24 is formed along just one edge of the partition 20. In such an example, the single projection 24 is approximately 2 mm in thickness in order to adjust a container 10 from 24 mm to 22 mm in width. Other dimensions are envisioned to fit any size of coverslip width.

In one aspect, the invention also includes a method for adjusting a coverslip container 10 to securely contain coverslips of varying lengths and/or widths by placement of one or more partitions 20 within the receptacle portion 11 of the container 10. For example, FIG. 7 shows that a partition 20 with projections 24 placed to fit 22 mm×40 mm coverslips, as shown by dashed lines, is replaceable with a partition 20 to form a receptacle area 11 to fit 24 mm×50 mm or 24 mm×60 mm coverslips.

If the container 10 is intended for shipping coverslips, the type of partition 20, as in flat or with projections 24, and placement of the partition(s) 20 are determined by the size of coverslips to be packaged. If the container 10 is made for more permanent use, such as for containing coverslips to be used in connection with automatic instruments, the container can be supplied with different partitions 20 to permit alteration of the receptacle portion 11 to receive a variety of coverslip sizes. For example, when switching from a 24×50 mm coverslip to a 22×40 mm coverslip, the user can remove the partition(s) 20 appropriate for holding 24×50 mm coverslips and replace with the partition(s) 20 with projections 24 appropriate for 22 mm width, positioning the partition(s) 20 in the correct position along the sidewalls 16 for 40 mm length.

The invention in its broadest aspects is not limited to the specific embodiments and details described herein. As such, departures may be made from the embodiments and details described herein without departing from the spirit and scope of the claims that follow.

I claim:

1. A container of the type used to contain coverslips comprising:
a base;
opposing side walls extending upwardly from and substantially perpendicular to said base; and
at least one partition perpendicular to said side walls and fitting securely against said sidewalls to adjust the length and/or width of said container.

2. The container of claim 1 further comprising opposing end walls extending upwardly from and substantially perpendicular to said base.

3. The container of claim 1 wherein said at least one partition may be repositioned to adjust the interior length of said container.

4. The container of claim 2 wherein said at least one partition may be repositioned to adjust the interior length of said container.

5. The container of claim 1 wherein said at least one partition further comprises projections located at or near the periphery of a set of opposing edges of said partition, said projections serving to adjust the internal width of said container.

6. The container of claim 2 wherein said at least one partition further comprises projections located at or near the periphery of a set of opposing edges of said partition, said projections serving to adjust the internal width of said container.

7. The container of claim 1 wherein the capacity of said container is at least 50 coverslips.

8. The container of claim 1 wherein the capacity of said container is approximately 500 coverslips.

9. A partition comprising:
a substantially flat plate with projections located at or near the periphery of a set of opposing edges of said partition,
wherein said partition is placed within a container for holding coverslips, and wherein said projections serve to adjust the internal width of said container.

10. The partition of claim 9 wherein the thickness of each of said projections is approximately 1 mm.

11. The partition of claim 9 wherein the total thickness of said projections is approximately 2 mm.
